(19) United States
(12) Patent Application Publication
(10) Pub. No.: US 2025/0114360 A1
(43) Pub. Date: Apr. 10, 2025

(54) NOVEL REGIMEN FOR AUGMENTING AND EXTENDING TRANSPLANT TOLERANCE

(71) Applicant: The Regents of the University of California, Oakland, CA (US)

(72) Inventors: Neil Kogut, Marina Del Rey, CA (US); Jeff Veale, Santa Monica, CA (US)

(73) Assignee: The Regents of the University of California, Oakland, CA (US)

(21) Appl. No.: 18/832,236

(22) PCT Filed: Jan. 31, 2023
(86) PCT No.: PCT/US2023/061682

§ 371 Date: Jul. 23, 2024

Related U.S. Application Data
(60) Provisional application No. 63/306,362, filed on Feb. 3, 2022.

(51) Int. Cl. A61K 31/517

(52) U.S. Cl. CPC A61K 31/517

(57) ABSTRACT

The invention disclosed herein provides methods and materials designed to improve organ transplantation outcomes. Embodiments of the invention include methods for administering the drug belumosudil to patients who have undergone an organ or other tissue transplantation (e.g., transplantation of a solid donor organ such as a kidney and/or receiving an infusion of donor stem cells such as CD34⁺ cells). In these methods, transplantation patients to whom belumosudil is administered are selected to be those not suffering from graft versus host disease (GVHD).

### CROSS REFERENCE TO RELATED APPLICATIONS

This application claims the benefit under 35 U.S.C. Section 119 (e) of co-pending and commonly-assigned U.S. Provisional Patent Application No. 63/306,362, filed Feb. 3, 2022, entitled “A NOVEL REGIMEN FOR AUGMENTING AND EXTENDING TRANSPLANT TOLERANCE”, which application is incorporated by reference herein.

### TECHNICAL FIELD

Embodiments of the disclosure concern at least the fields of medicine, transplantation, immunology and pharmacology.

### BACKGROUND OF THE INVENTION

Advances in surgical techniques and improved drugs that prevent infection and rejection have allowed transplantation of solid organs to become an effective treatment for many diseases. Transplanted organs include heart, intestine, liver, lung, pancreas and kidney. Kidney transplantation, or renal transplantation, is the organ transplant of a kidney into a patient with end-stage renal disease. Kidney transplantation can be classified as deceased or living-donor transplantation, and may further be classified according to the degree of relationship between donors and recipients, as related or non-related, and according to the number of human leukocyte antigen (HLA)-matches or HLA-mismatches.

The indication for kidney transplantation is end-stage renal disease (ESRD, clinically defined as a glomerular filtration rate≤15), regardless of the primary cause, defined as a glomerular filtration rate below a pre-determined level. Common diseases leading to ESRD include malignant hypertension, infections, diabetes mellitus, and focal segmental glomerulosclerosis; genetic causes include polycystic kidney disease, a number of inborn errors of metabolism, and autoimmune conditions such as lupus. Diabetes is a common cause of kidney transplantation, accounting for approximately 25% of those in the US. The majority of renal transplant recipients are on dialysis at the time of transplantation.

The major barrier to organ transplantation between genetically non-identical patients lies in the recipient's immune system, which can respond to the transplanted kidney as “non-self” and reject it. Thus, having medications to suppress the immune system is essential, however, suppressing an individual's immune system places that individual at greater risk of infection and cancer, in addition to the side effects of the medications. Recipients usually receive a mixture of three maintenance immunosuppressive drugs, including agents such as cyclosporine, tacrolimus or sirolimus; prednisone; and an inhibitor of nucleic acid synthesis such as mycophenolate mofetil. These medications have side effects that include malignancy, infection, diabetes, heart disease, and ironically nephrotoxicity that can impair renal allograft function leading to graft loss. In spite of modern immunosuppressive drugs, in some centers acute rejection can occur in 10-25% of people after transplant.

Generally transplant recipients will have to take immunosuppressive anti-rejection drugs for as long as the transplanted organ (e.g. kidney) functions. Even for a mixture of widely used immunosuppressives the cost can be high. For this reason, the achievement of transplantation tolerance is highly desirable. In this context, “transplantation Tolerance” may be defined as survival of the grafted tissue/organ in the recipient's immune system without causing rejection, while maintaining host immune response intact and performing normal function without requiring immunosuppressive medications

Preclinical studies have shown that conditioning with total lymphoid irradiation (TLI) and anti-thymocyte globulin (ATG) is advantageous for inducing transplantation tolerance after combined organ and bone marrow transplantation because the conditioning regimen (one typically given after the kidney transplant but before the hematopoietic stem cell transplant) prevents graft versus host disease (GVHD) as compared to total body irradiation (TBI). For a review, see Strober et al. (2011) Seminars in Immunology 23:273-281.

It is of great clinical interest to develop therapeutic regimens for organ transplantation that can achieve greater tolerance, for example so as to diminish the need for immunosuppressive drugs in recipient patients.

### SUMMARY OF THE INVENTION

As discussed in detail below, the invention disclosed herein provides methods and materials designed to improve organ transplantation outcomes. Embodiments of the invention include methods for administering the drug belumosudil to patients who have undergone an organ transplantation. In these methods, the transplant patients to whom belumosudil is administered are selected to be those who have not been diagnosed with graft versus host disease (GVHD). For example, in certain embodiments of the invention, the timing of belumosudil administration is selected to be prior to the typical time of development of GVHD in transplant patients, for example within the first three months following organ transplantation (as acute classic GVHD typically presents within 100 days of transplantation). Illustrative embodiments of the invention include the administration of belumosudil in conjunction with a transplantation conditioning regimen of anti-thymocyte globulin (rATG) and total lymphoid irradiation (TLI) to induce transplant tolerance in patients with end stage renal disease who are undergoing combined kidney and hematopoietic stem cell transplantation. Optimally, belumosudil is administered to such patients on day 28 post transplantation.

Belumosudil (also known as KD025) is an orally available Rho-associated coiled-coil kinase 2 (ROCK2) selective inhibitor that is FDA approved for the treatment of steroid refractory chronic graft vs host disease following allogeneic HSCT for hematological malignancies. In vitro data have demonstrated that belumosudil (1) attenuates IL-21 and IL-17 secretion in human CD4+ T cells via STAT3, IRF4 and RORγt regulation, and (2) leads to increased percentages of Foxp3+ CD4+ T cells via a STAT5-dependent mechanism and upregulates the suppressive function of human regulatory T cells (Tregs). These mechanisms of action suggest that belumosudil modulates immune homeostasis by shifting the Th17/Treg balance towards a Treg phenotype. Since Belumosudil increases endogenous Tregs, it obviates the need for the isolation and expansion of exogenous Tregs which is a component of a number of other transplant tolerance trials.

As disclosed herein, the increase in regulatory T cell number and function brought on by belumosudil administered according to the methods of the invention will augment and prolong donor chimerism following, for example, combined kidney and hematopoietic stem cell transplantation conditioned with rATG/TLI, and will further allow for successful withdrawal of immunosuppression while preserving graft function. Whereas rATG/TLI regimen alone has been effective in allowing cessation of anti-rejection medications following HLA matched living donor transplants, it has not led to transplant tolerance in recipients of HLA single haplotype matched transplants. Embodiments of the invention that combine the administration of belumosudil with rATG/TLI can lead to the successful application of transplant tolerance protocols to recipients of HLA single haplotype and mismatched living donor transplants and ultimately deceased donor transplants, thereby extending the benefits of transplant tolerance to the vast majority (77%) of recipients who receive allografts from deceased donors. Concomitantly, embodiments of this invention allow use of this approach to transplant recipients of other solid organs (e.g. liver and heart etc.) as well composite vascularized allografts (e.g. face, hand, etc.). Lastly, it is anticipated that this approach will be safer than more intensive transplant tolerance regimens utilizing chemotherapeutic agents and radiation as there will be less risk of graft vs host disease.

Embodiments of the invention include methods for administering belumosudil to patients who have undergone tissue and/or organ transplantation. Typically, these methods for administering belumosudil comprise the steps of selecting a patient for belumosudil administration, wherein the patent is selected as one having had or selected for a transplantation procedure with one or more genetically non-identical cells, tissues or organs; and then administering belumosudil to the patient, wherein the belumosudil is administered following transplantation and prior to the onset of graft versus host disease in the patient (i.e., this embodiment of the therapeutic methodology is designed for patients who are not suffering from GVHD). In these methods of the invention, belumosudil can be administered within 3 months of the patient being transplanted with the one or more genetically non-identical cells, tissues or organs, for example the transplantation of a solid organ such as a kidney and/or the infusion of stem cells such as CD34⁺ cells. In one such illustrative embodiment of the invention, belumosudil can be administered within one month (e.g. 28 days) of the patient being transplanted with the one or more genetically non-identical tissues. Typically in these methods, the administration of belumosudil to the patient occurs in conjunction with and following a transplantation conditioning regimen comprising anti-thymocyte globulin (rATG) administration and total lymphoid irradiation (TLI).

The methods of the invention can be used in a variety of transplantation procedures such as combined kidney and hematopoietic stem cell transplantation. In illustrative embodiments of the invention, the one or more genetically non-identical cells, tissues or organs comprises at least one of: hematopoietic stem cells, kidney cells, heart cells, intestinal cells, liver cells, lung cells or pancreatic cells. In some embodiments of the invention, the one or more genetically non-identical cells, tissues or organs are obtained from a human lymphocyte antigen (HLA) single haplotype-matched donor (as is known in this area of technology, such donors can include deceased donors). In other embodiments of the invention, the one or more genetically non-identical cells, tissues or organs are obtained from a 0-3 antigen HLA mismatched donor (e.g., 0-3 HLA mismatches occurring at least one of: HLA-A, HLA-B, HLA-C or HLA-DR).

Other objects, features and advantages of the present invention will become apparent to those skilled in the art from the following detailed description. It is to be understood, however, that the detailed description and specific examples, while indicating some embodiments of the present invention, are given by way of illustration and not limitation. Many changes and modifications within the scope of the present invention may be made without departing from the spirit thereof, and the invention includes all such modifications.

### BRIEF DESCRIPTION OF THE DRAWINGS

FIG. 1: Schematic showing structure of Belumosudil. Belumosudil is in the class of drugs known as serine/threonine kinase inhibitors. Specifically, it is an inhibitor of Rho-associated coiled-coil kinase 2 (ROCK2; ROCK-II). Belumosudil binds to and inhibits the serine/threonine kinase activity of ROCK2

### DETAILED DESCRIPTION OF THE INVENTION

In the description of embodiments, reference may be made to illustrative embodiments by which the invention may be practiced. It is to be understood that other embodiments may be utilized, and structural changes may be made without departing from the scope of the present invention.

Approximately 20,000 kidney transplants are performed in the United States annually. Although kidney transplantation prolongs survival and improves the quality of life of patients with end-stage renal disease, it has two major limitations: the risk of graft rejection and the need for lifelong immunosuppression. Conventional immunosuppression following kidney transplantation is associated with serious, long-term complications, including infection, secondary malignancies, hypertension, hyperlipidemia, diabetes, and cardiovascular disease. Calcineurin inhibitors may damage kidney function and contribute to graft failure. In addition, the intensive and complicated pharmaceutical regimen adversely affects quality of life and leads to a risk of non-compliance. Moreover, the costs of conventional immunosuppression are high, and ironically contribute to graft failure. In spite of the immunosuppression that is achieved via agents such as tacrolimus, corticosteroids, and mycophenolate, most kidney grafts eventually fail. According to recent data from the Organ Procurement and Transplant Network, the graft half-life following living donor transplantation treated with conventional immunosuppression is approximately 15 years. Following graft failure, patients must return to dialysis and/or undergo re-transplantation.

With the twin aims of prolonging graft survival and eliminating (or at least minimizing) immunosuppression, investigators in the field of transplantation have long sought to find a reliable and safe method to induce transplant tolerance (defined as a state of immunological non-responsiveness to a transplanted organ in the absence of immunosuppression). Researchers at Mass General, Northwestern and Stanford have published reports going back decades of successful withdrawal of immunosuppression after combined living donor kidney and hematopoietic stem cell transplant. Each center has adopted its own approach, utilizing conditioning regimens of varying intensity which lead to different levels and duration of chimerism. The investigators at Mass General have studied various iterations of a non-myeloablative conditioning regimen with the goal of transient mixed chimerism. In comparison, Northwestern seeks to achieve full chimerism through a more aggressive conditioning regimen akin to a reduced intensity haploidentical HSCT with an infusion of a cell product enriched for “facilitating cells”. Somewhat intermediate, the objective at Stanford is stable mixed chimerism following a regimen of TLI/ATG with an infusion of a defined dose of CD34 and CD3 cells.

We have recently opened an immune tolerance protocol (IND 18438) based on the Stanford regimen. In our opinion, the Stanford platform offers several advantages. One, it is safe and well tolerated. In contrast to the Northwestern regimen, there have been no cases of graft vs host disease nor serious infections. Second, in the HLA matched living donor setting, the results have been impressive with approximately 80% of patients being able to stop immunosuppression at one year and enjoy continued freedom from immunosuppression with excellent graft function (in some cases over 10 years later). Third, it is easily adapted to deceased donor transplantation which is where the field of transplant tolerance is ultimately headed.

However, a drawback of the Stanford regimen is that the same results have not been seen in the HLA mismatched setting. In this group, the Stanford researchers noted loss of chimerism and a number of graft rejections when immunosuppression was stopped at one year. In an attempt to achieve higher and more prolonged chimerism, they escalated the dose of the CD34 and CD3 cells. Although reduction of immunosuppression to single agent tacrolimus has been achieved in some patients, complete withdrawal of immunosuppression has not been possible. Therefore, there is a need to develop other approaches to reach higher and more sustained levels of chimerism.

In the last several years, interest has grown in utilizing regulatory T cells for the prevention and/or treatment of graft rejection following kidney transplant. A number of trials have demonstrated the feasibility of collecting and isolation exogenous T regs, either polyclonal or donor specific. Infusion of these cells has been safe and well tolerated. There are insufficient data to determine efficacy at this point and clinical trials are underway to further study the role of T regs as a therapeutic strategy following kidney transplantation. More recently, there has been a trend to use T regs in conjunction with combined HSCT/KT in what is sometimes referred to as combinatorial cell therapy. A small study in primates demonstrated some efficacy with this approach in allowing immunosuppression withdrawal. This approach is now the focus of a clinical trial being conducted jointly by Stanford and Northwestern in which T regs are given to recipients shortly after the combined HSCT/KT conditioned with TLI/ATG (see ClinicalTrials.gov Identifier: NCT03943238).

The collection, isolation, and expansion of exogenous T regs requires considerable laboratory space, time, resources, and expertise. In contrast, a pharmacological approach designed to endogenously increase the number and function of T regs in vivo obviates the need for the complex process for producing exogenous T regs and might also allow for more prolonged expansion of the T reg compartment. A number of drugs raise T regs including IL-2 and mammalian target of rapamycin (mTOR) inhibitors. However, IL-2 requires a daily injection and may cause constitutional side effects such as fatigue and flu-like symptoms. In some studies, use of mTOR inhibitors may cause a number of significant side effects such as hyperlipidemia, thrombotic microangiopathy, impaired wound healing, edema, and mouth ulcers.

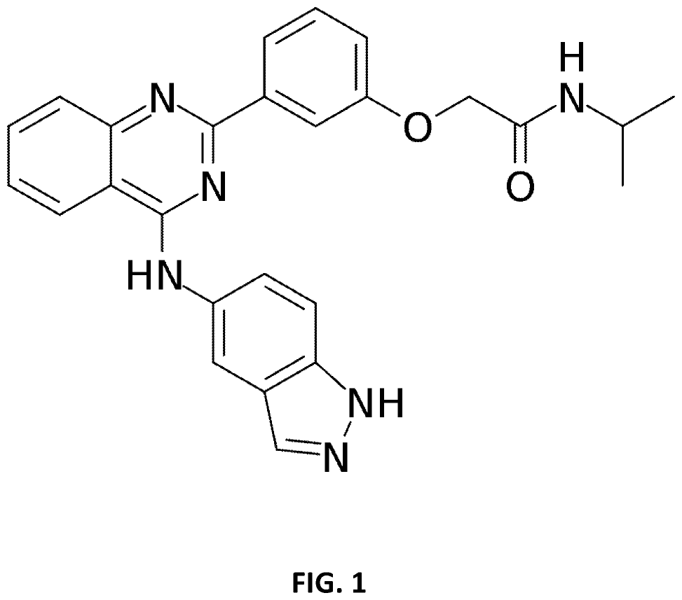

In contrast, the investigational Rock-2 inhibitor belumosudil (KD025, FIG. 1)) demonstrates several properties which make it an attractive agent for our protocol. Belumosudil increases T reg number and function and shift in balance of effector/regulatory cells towards the latter through augmented stat 5 phosphorylation. Belumosudil reduces IL-17 and IL-21 through reduction in stat 3 phosphorylation. These inflammatory cytokines may play a role in graft rejection. Belumosudil down-regulates key fibrotic processes in preclinical models, including profibrotic gene transcription, stress fiber formation, myofibroblast activation and collagen deposition. Interstitial fibrosis is a prominent histological feature of chronic graft rejection. In July 2021 the FDA approved Belumosudil for the treatment of chronic graft vs host disease after failure of two prior lines of treatment. Several recent reports of a large number of patients with refractory chronic graft vs host disease treated with belumosudil have demonstrated the safety and efficacy of the drug. For further discussions of belumosudil, see, e.g. PCT Publications: WO2006105081; WO2008054599; WO2010104851; WO2014055996 US Patent Publication Nos. 20170112832, 20160237095 and 20150238601; and also Boerma et al., Blood Coagul Fibrinolysis, 2008, 19(7): 709-718, the contents of each of which is incorporated herein by reference.

Embodiments of the invention include methods for administering the drug belumosudil to patients who are scheduled for, are undergoing, or have undergone tissue and/or organ transplantation procedure. Typically, these methods for administering belumosudil comprise the steps of selecting a patient for belumosudil administration, wherein the patent is selected as one scheduled for a transplantation procedure, one undergoing a transplantation procedure, or one who has undergone a transplantation procedure with one or more genetically non-identical cells, tissues or organs from a living or deceased donor (see, e.g. U.S. Patent Publication No. US 2021-0189344, the contents of which are incorporated by reference); and then administering belumosudil to the patient, wherein the belumosudil is administered following the transplantation procedure and prior to the onset of graft versus host disease in the patient (i.e. prior to the onset of graft versus host disease that may occur from the transplanted cells used in that specific transplantation procedure).

Typically in these methods, the administration of belumosudil to the patient occurs in conjunction with a transplantation conditioning regimen comprising anti-thymocyte globulin (rATG) administration and total lymphoid irradiation (TLI). In these methods of the invention, belumosudil is administered within 3 months of the patient being transplanted with the one or more genetically non-identical cells, tissues or organs. For example, in such methods belumosudil can be administered within one month (e.g. 28 days) of the patient being transplanted with the one or more genetically non-identical cells, tissues or organs.

The methods of the invention can be used in a variety of transplantation procedures such as combined kidney and hematopoietic stem cell transplantation. In illustrative embodiments of the invention, the one or more genetically non-identical cells, tissues or organs comprises at least one of: hematopoietic stem cells, kidney cells, heart cells, intestinal cells, liver cells, lung cells or pancreatic cells. Moreover, certain embodiments of the invention are used in vascular composite allografts. Vascular composite allografts involve the transplantation of multiple structures that may include skin, bone, muscles, blood vessels, nerves and connective tissue. Face and hand transplants are the most well-known types of vascular composite allografts. The use of vascular composite allografts transplantation is increasing as this technology matures and further includes, for example, the transplantation of reproductive organs.

In certain embodiments of the invention, one or more genetically non-identical cells, tissues or organs utilized for a transplantation procedure comprise at least one of: hematopoietic stem cells, kidney cells, heart cells, intestinal cells, liver cells, lung cells, pancreatic cells, face cells, hand cells, or reproductive organ cells. In some embodiments of the invention, the one or more genetically non-identical cells, tissues or organs consists essentially of hematopoietic stem cells. In some embodiments of the invention, the one or more genetically non-identical cells, tissues or organs consists essentially of kidney cells. In some embodiments of the invention, the one or more genetically non-identical cells, tissues or organs consists essentially of hematopoietic stem cells and kidney cells. In certain embodiments of the invention, the patient treated according to the methods disclosed herein is selected to be one who has not undergone a previous transplantation procedure with one or more genetically non-identical cells, tissues or organs. In other embodiments of the invention, the patient treated according to the methods disclosed herein is selected to be one who has been administered one more immunosuppressive agents comprises cyclosporine, tacrolimus, sirolimus; prednisone; or an inhibitor of nucleic acid synthesis.

When transplanting tissues, medical practitioners consider HLA (human leukocyte antigen) matching in order to optimize the chances of a successful transplantation procedure. HLAs are proteins covering the surface of most cells, and these proteins dictate an individual's tissue type. Each parent donates a set of HLAs to their children, and medical practitioners attempt to match the recipient and donor HLA types before a transplantation procedure. The degree of human leukocyte antigen (HLA) matching between the transplant recipient and donor tissues has a significant impact on the outcomes of both related and unrelated donor tissue transplantation. As there are six total HLA antigens expressed on the surface of cells, matches are rated on a scale from 1 to 6. When a donor is a 6 out of 6 match, there is relatively small chance for the emergence of graft-related disease and other associated complications in the recipient. The greater the number of HLA mismatches, the greater the risk of post transplantation complications.

In some embodiments of the invention, the one or more genetically non-identical cells, tissues or organs comprise at least one human leukocyte antigen that is mismatched with a human leukocyte antigen present in the patient. In other embodiments of the invention, the one or more genetically non-identical cells, tissues or organs comprise at least 2, 3, 4, 5 or 6 human leukocyte antigens that are mismatched with human leukocyte antigens present in the patient. Typically, for example, such HLA mismatches occur at least one of: HLA-A, HLA-B, HLA-C or HLA-DR antigens. While in some embodiments of the invention, the one or more genetically non-identical cells, tissues or organs are obtained from a human lymphocyte antigen (HLA) single haplotype-matched donor, in other embodiments of the invention, the one or more genetically non-identical cells, tissues or organs are obtained from a 0-3 antigen HLA mismatched donor.

Embodiments of the present invention utilize pharmaceutically acceptable compositions which comprise a therapeutically-effective amount of belumosudil formulated together with one or more pharmaceutically acceptable carriers (additives) and/or diluents. The phrase “therapeutically-effective amount” as used herein means that amount of a compound, material, or composition comprising belumosudil of the present invention which is effective for producing some desired therapeutic effect in at least a sub-population of cells in a patient at a reasonable benefit/risk ratio applicable to any medical treatment, e.g. reasonable side effects applicable to any medical treatment. Embodiments of the invention include Belumosudil for use in the treatment of graft versus host disease prior to the onset of graft versus host disease. Similar embodiments of the invention include Belumosudil for use in inhibiting the onset of graft versus host disease.

The phrase “pharmaceutically acceptable” is employed herein to refer to those compounds, materials, compositions, and/or dosage forms which are, within the scope of sound medical judgment, suitable for use in contact with the tissues of human beings and animals with toxicity, irritation, allergic response, or other problems or complications, commensurate with a reasonable benefit/risk ratio. The phrase “pharmaceutically-acceptable carrier” as used herein means a pharmaceutically-acceptable material, composition or vehicle, such as a liquid or solid filler, diluent, excipient, manufacturing aid (e.g., lubricant, talc, magnesium, calcium or zinc stearate, or steric acid), or solvent encapsulating material, involved in carrying or transporting the subject compound from one organ, or portion of the body, to another organ, or portion of the body. Each carrier must be “acceptable” in the sense of being compatible with the other ingredients of the formulation and not injurious to the patient.

Actual dosage levels of belumosudil in the pharmaceutical compositions of this invention may be varied so as to obtain an amount of the active ingredient which is effective to achieve the desired therapeutic response for a particular patient, composition, and mode of administration, without being toxic to the patient. The selected dosage level can depend upon a variety of factors including the activity of the particular compound of the present invention employed, the route of administration, the time of administration, the rate of excretion or metabolism of the particular compound being employed, the rate and extent of absorption, the duration of the treatment, other drugs, compounds and/or materials used in combination with the particular compound employed, the age, sex, weight, condition, general health and prior medical history of the patient being treated, and like factors well known in the medical arts.

Using the disclosure provided herein, a physician having ordinary skill in the art can readily determine and prescribe the effective amount of the pharmaceutical belumosudil composition used (e.g., 200 mg QD, 200 mg BID, 400 mg QD and the like). For example, the physician or veterinarian could start doses of the compounds of the invention employed in the pharmaceutical composition at levels lower than that required in order to achieve the desired therapeutic effect and gradually increase the dosage until the desired effect is achieved.

In general, a suitable daily dose of a compound of the invention will be that amount of the compound which is the lowest dose effective to produce a therapeutic effect. Such an effective dose will generally depend upon the factors described above. Generally, oral doses of the compounds of this invention for a patient, when used for the indicated effects, will range from about 0.0001 to about 100 mg per kilogram of body weight per day.

In certain embodiments, a dose of a compound or a composition is administered to a subject every day, every other day, every couple of days, every third day, once a week, twice a week, three times a week, or once every two weeks. If desired, the effective daily dose of the active compound may be administered as two, three, four, five, six or more sub-doses administered separately at appropriate intervals throughout the day, optionally, in unit dosage forms. In some embodiments, a dose(s) of a compound or a composition is administered for 2 days, 3 days, 5 days, 7 days, 14 days, or 21 days. In certain embodiments, a dose of a compound or a composition is administered for 1 month, 1.5 months, 2 months, 2.5 months, 3 months, 4 months, 5 months, 6 months or more.

In one illustrative embodiment of the invention involving kidney transplantation, subjects can receive (1) an intravenous infusion of GCSF-mobilized, Miltenyi-enriched CD34+ cells (e.g. at least 8×10⁶ cells per kilogram, target ≥10×10⁶ cells per kilogram) followed by (2) an infusion of CD3+ cells (at least 10×10⁶, target ≥100×10⁶ cells per kilogram) from an HLA single haplotype-matched related or HLA mismatched unrelated living donor after a conditioning regimen consisting of TLI and rATG. Subjects can receive 200 mg belumosudil daily starting on day 28 after transplant for duration of protocol. For transplant and Treatment, the conditioning regimen followed by the infusion of CD34+ and CD3+ cells from an HLA single haplotype-matched related or 0-3 antigen (at A,B,C,DR) HLA mismatched unrelated living donor can take place over 12 days post-kidney transplant. Belumosudil can be taken starting at day 28 and can continue through 24 months post-kidney transplant. Follow-up: Subjects can be followed after belumosudil is stopped for 12 months. Patient endpoints observed in studies of such methods include percentage patient and kidney allograft survival at one year; percentage of subjects that develop kidney allograft rejection based on clinical parameters and/or kidney biopsy; percentage of subjects that develop acute and/or chronic graft vs host disease; percentage of subjects that experience grade 3 or 4 toxicities attributable to total lymphoid irradiation; percentage of subjects that experience grade 3 or 4 toxicities attributable to study drug; percentage of subjects that require delay or interruption of study drug and/or cessation of study drug due to toxicity; percentage of subjects with ≥1% donor chimerism in one or more leukocyte subsets (T cells, B cells, NK cells, and granulocytes) at 3 months, 6 months, 12 and 18 months and absolute level of chimerism at the same time points; percentage change of regulatory T cells and other leukocyte subsets at 3, 6, 12, and 18 months and absolute number of leukocyte subsets at the same time points; percentage of subjects successfully withdrawn from mycophenolate at 12 months and tacrolimus at 18 months; percentage of subjects needing to resume mycophenolate and/or tacrolimus due to loss of chimerism and/or development of graft rejection or graft vs host disease; and time to loss of chimerism and/or graft rejection after cessation of mycophenolate and tacrolimus.

A summary of one illustrative treatment plan is as follows. Immediately after living donor kidney transplantation, subjects can begin a conditioning regimen of rATG (1.5 mg/kg IV daily×5 days) and total lymphoid irradiation (1200 cgy in 10 divided fractions). An infusion of at least 8×10⁶ (target ≥10×10⁶) donor CD34 cells/kg recipient weight and of at least 10×10⁶ (target 100×10⁶) donor CD3 cells/kg recipient weight can then be given. A triple immunosuppressive regimen of tacrolimus, corticosteroids, and mycophenolate can be utilized. Tacrolimus can be given on a tapering schedule from day 1 through month 18. Corticosteroids can be given on a tapering schedule from day 0 through the first four months. Mycophenolate can be given at a fixed dose from day 11 through month 12. Subjects can receive belumosudil 200 mg by mouth daily from day 28 following the kidney transplant and continued through month 24. At serial time points, (1) chimerism can be measured in recipient whole blood and leukocyte subsets (2) graft function can be monitored (3) protocol biopsies of the graft can be obtained and (4) a T cell panel including regulatory T cells can be performed. Mycophenolate can be stopped at 12 months and tacrolimus can be stopped at 18 months if all of the following conditions are met: chimerism (defined as ≥1% donor type cells among the T cells, B cells, NK cells, and granulocytes) is detectable; stable graft function; absence of clinical or pathological evidence of acute rejection; and absence of clinical or pathological evidence of graft vs host disease. Immunosuppression taper can be stopped and/or immunosuppression can be resumed for any of the following conditions: loss of chimerism; clinical or pathological evidence of acute rejection and/or clinical or pathological evidence of graft vs host disease.

As noted above, embodiments of the invention include a conditioning regimen of rATG. Briefly, rATG (brand name Thymoglobulin) is an FDA-approved purified globulin preparation obtained from the serum of rabbits that have been immunized to human thymocytes for induction and rejection therapy in kidney transplantation. It is indicated for the prophylaxis and treatment of acute rejection in patients receiving a kidney transplant. In embodiments of the invention, subjects can receive a total of 5 intravenous doses of rATG administered through a central venous line. Each dose can be 1.5 mg/kg. rATG can be administered on the day of transplantation (Day 0) and on Days 1, 2, 3, and 4 post-transplant in the inpatient setting. rATG can be held for severe neutropenia (ANC</=500) and/or thrombocytopenia (platelets </=20,000) and can be resumed when the ANC and/or platelet count exceed these levels. Subjects with absolute neutrophil count≤500/μL can receive GCSF 300 μg/kg daily for 3 days. A delay in administration of rATG can require cancelling discharge scheduled for day 4 and the subject can remain an inpatient until the last dose of rATG has been administered.

As noted above, embodiments of the invention include total lymphoid irradiation (TLI). In illustrative embodiments, subjects can receive ten treatments of fractionated irradiation (120 cGy each) targeted to the lymph nodes, spleen and thymus gland on Days 1 through 4 and 7 through 11 after transplantation. The total dose can be 1200 cGy and can be given to the inverted Y, paraortic, and mantle fields. TLI can be administered using a megavoltage medical linear accelerator—such as the TrueBeam Radiotherapy Delivery System (Varian Medical Systems, Inc.)—in the Department of Radiation Oncology at UCLA, which is intended to provide stereotactic radiosurgery and precision radiotherapy for lesions, tumors, and conditions anywhere in the body where radiation treatment is indicated. The linear accelerator can be used off label for an unapproved indication (kidney transplant) in this study. The team can make its best effort to prevent treatment breaks due to technical factors such as machine downtime. If the primary treatment machine is down, the team can make its best effort to treat the patient on a similar alternate machine. During the administration of TLI, subjects can be monitored for the development of neutropenia (absolute neutrophil count≤500/μL), thrombocytopenia (platelets <20,000/μL), and infection. TLI can not be interrupted for neutropenia. Subjects with absolute neutrophil count≤500/μL can receive GCSF 300 μg/kg daily for 3 days. TLI can be held for platelet count≤20,000/μL. It can also be held for neutropenic fever and/or infection. TLI can be resumed when platelet count recovers to above 20,000/μL and/or neutropenic fever and/or infection resolve.

As noted above, embodiments of the invention include administration of tacrolimus, (brand name PROGRAF) an FDA-approved calcineurin-inhibitor immunosuppressant used to prevent organ rejection in patients receiving allogeneic kidney transplants. It can be given without taper for at least 3 full months, during which the target trough level can be 10-12 ng/ml. The initial dose can be 0.075 mg/kg BID with dose adjustments for target trough. Starting in Month 4, tacrolimus can be tapered as per a schedule. Subjects with chimerism at the start of Month 6 can continue to follow a taper schedule.

### REFERENCES

1. Busque, S., et al., Mixed chimerism and acceptance of kidney transplants after immunosuppressive drug withdrawal. Sci Transl Med, 2020. 12(528).
2. Scandling, J. D., et al., Chimerism, graft survival, and withdrawal of immunosuppressive drugs in HLA matched and mismatched patients after living donor kidney and hematopoietic cell transplantation. Am J Transplant, 2015. 15(3): p. 695-704.
3. Ferreira, L. M. R., et al., Next-generation regulatory T cell therapy. Nat Rev Drug Discov, 2019. 18(10): p. 749-769.
4. Cutler C S., et al., Belumosudil for Chronic Graft-versus-Host Disease (cGVHD) After 2 or More Prior Lines of Therapy: The ROCKstar Study. Blood. 2021 Dec. 2; 138(22): 2278-2289. doi: 10.1182/blood.2021012021. Erratum in: Blood. 2022 Mar. 17; 139(11): 1772. PMID: 34265047; PMCID: PMC8641099.
5. Jagasia M, Lazaryan A, Bachier C R, Salhotra A, Weisdorf D J, Zoghi B, Essell J. Green L, Schueller O, Patel J, Zanin-Zhorov A, Weiss J M, Yang Z, Eiznhamer D, Aggarwal S K, Blazar B R, Lee S J. ROCK2 Inhibition With Belumosudil (KD025) for the Treatment of Chronic Graft-Versus-Host Disease. J Clin Oncol. 2021 Jun. 10; 39(17):1888-1898. doi: 10.1200/JCO.20.02754. Epub 2021 Apr. 20. PMID: 33877856; PMCID: PMC8189612.
6. Issa F, et al The Fourth International Workshop on Clinical Transplant Tolerance. Am J Transplant. 2021 Jan. 21(1): 21-31. doi: 10.1111/ajt.16139. Epub 2020 Jul. 22. PMID: 32529725.
7. Zanin-Zhorov A, Blazar B R. ROCK2, a critical regulator of immune modulation and fibrosis has emerged as a therapeutic target in chronic graft-versus-host disease. Clin Immunol. 2021 September; 230:108823. doi: 10.1016/j.clim.2021.108823. Epub 2021 Aug. 14. PMID: 34400321; PMCID: PMC8456981.

All publications mentioned herein (e.g. those disclosed above) are incorporated by reference to disclose and describe aspects, methods and/or materials in connection with the cited publications. Many of the techniques and procedures described or referenced herein are well understood and commonly employed by those skilled in the art. Unless otherwise defined, all terms of art, notations and other scientific terms or terminology used herein are intended to have the meanings commonly understood by those of skill in the art to which this invention pertains. In some cases, terms with commonly understood meanings are defined herein for clarity and/or for ready reference, and the inclusion of such definitions herein should not necessarily be construed to represent a substantial difference over what is generally understood in the art.

1. A method of administering belumosudil comprising:
(a) selecting a patient for belumosudil administration, wherein the patent is selected for a transplantation procedure with one or more genetically non-identical cells, tissues or organs; and
(b) administering belumosudil to the patient, wherein the belumosudil is administered following transplantation and prior to the diagnosis or onset of graft versus host disease in the patient.

2. The method of claim 1, wherein the administration of belumosudil to the patient occurs in conjunction with a transplantation conditioning regimen comprising anti-thymocyte globulin (rATG) administration and total lymphoid irradiation (TLI).

3. The method of claim 1, wherein belumosudil is administered within 3 months of the patient being transplanted with the one or more genetically non-identical cells, tissues or organs.

4. The method of claim 3, wherein the belumosudil is administered within one month of the patient being transplanted with the one or more genetically non-identical cells, tissues or organs.

5. The method of claim 1, wherein the one or more genetically non-identical cells, tissues or organs are obtained from a deceased donor.

6. The method of claim 1, wherein the one or more genetically non-identical donor tissues or organs comprise at least one human leukocyte antigen that is mismatched with at least one human leukocyte antigen present in the patient.

7. The method of claim 1, wherein the one or more genetically non-identical donor tissues or organs comprise at least 2, 3, 4, 5 or 6 human leukocyte antigens that are mismatched with human leukocyte antigens present in the patient.

8. The method of claim 7, wherein the HLA mismatches occur at least one of: HLA-A, HLA-B, HLA-C or HLA-DR.

9. The method of claim 1, wherein the one or more genetically non-identical cells, tissues or organs comprise at least one of: hematopoietic stem cells, kidney cells, heart cells, intestinal cells, liver cells, lung cells, pancreatic cells, face cells, hand cells, or reproductive organ cells.

10. The method of claim 1, wherein the one or more genetically non-identical cells, tissues or organs comprise at least two of: hematopoietic stem cells, kidney cells, heart cells, intestinal cells, liver cells, lung cells, pancreatic cells, face cells, hand cells, or reproductive organ cells.

11. The method of claim 1, wherein the one or more genetically non-identical cells, tissues or organs consists essentially of hematopoietic stem cells.

12. The method of claim 1, wherein the one or more genetically non-identical cells, tissues or organs consists essentially of kidney cells.

13. The method of claim 1, wherein the patient has not undergone a previous transplantation procedure with one or more genetically non-identical cells, tissues or organs.

14. The method of claim 13, wherein the patient has been administered one more immunosuppressive agents comprises cyclosporine, tacrolimus, sirolimus; prednisone; or an inhibitor of nucleic acid synthesis.

15. Belumosudil for use in the treatment of graft versus host disease prior to the onset of graft versus host disease.
